(19) United States
(12) Patent Application Publication
(10) Pub. No.: US 2012/0093876 A1
(43) Pub. Date: Apr. 19, 2012

(54) Ophthalmic Formulations, Methods Of Manufacture And Methods of Normalizing Meibomian Gland Secretions

(75) Inventors: George W. Ousler, III, North Andover, MA (US); Matthew J. Chapin, Amesbury, MA (US); Mark B. Abelson, Andover, MA (US); George Minno, Windham, NH (US); Aron Shapiro, Somerville, MA (US)

(73) Assignee: Aciex, Inc., Westborough, MA (US)

(21) Appl. No.: 12/903,881

(22) Filed: Oct. 13, 2010

(51) Int. Cl. A61K 31/65; A61P 27/02; A61K 9/00

(52) U.S. Cl. 424/400; 514/152

(57) ABSTRACT

The present invention provides process for producing non-aqueous compositions for normalizing meibomian gland secretions. The present invention further provides compositions and methods for treating and/or preventing the signs and/or symptoms of dry eye disease.

### FIELD OF THE INVENTION

The present invention relates generally to novel compositions and methods for normalizing meibomian gland secretion and the treatment and prevention of diseases related thereto. More specifically, the present invention relates to ophthalmic compositions comprising an anti-infective agent useful for the normalization of abnormal meibomian gland secretions. The invention additionally relates to methods of administering such compositions to a subject in need thereof.

### BACKGROUND

Tears are comprised of three layers. The mucus layer coats the cornea forming a foundation so the tear film can adhere to the eye. The middle aqueous layer provides moisture and supplies oxygen and other important nutrients to the cornea. The outer lipid layers is an oily film that seals the tear film on the eye and helps to prevent evaporation of the layers beneath. Meibomian glands (located at the lid margins) are primarily responsible for lipid generation, and abnormal secretions from in these glands can lead to an unhealthy lipid layer in the tear film. The lipid secreted by the meibomian glands also retards evaporation from the preocular surface, lowers the surface tension of tears, prevents spillover of tears from the lid margin, prevents the contamination of the tear film by sebaceous lipids and prevents damage to the skin of the lid margin.

Abnormal meibomian gland secretions, a condition associated with obstruction and inflammation of the meibomian glands, is a widespread and chronic problem. Abnormal meibomian secretions is one of the most common causes of dry eye syndrome.

The signs and symptoms of dry eye are exacerbated by abnormalities in the lipid layer of the tear film, which is produced by the meibomian glands. Obstruction of the meibomian ducts causes accumulation of meibomian gland secretions, known as meibum. Accumulation of meibum within the meibomian gland can lead to inflammation of the gland and bacterial colonization. The colonizing bacteria have lipases that break the non-polar wax and sterol esters into triglycerides and free fatty acids (polar lipids), thus altering the normal composition of the meibum and the lipid layer of the tear film causing the tear film to become unstable, and the surface of the eye unwettable. Another potential mechanism of diseases associated with abnormal meibomian gland secretion is through quorum sensing, a means which by bacteria communicate with each other. When the normal bacterial flora balances change different genes in the bacteria are transcribed producing immune stimulating compounds. This leads to inflammation of the meibomian gland and altered secretions and gland obstruction.

Currently, there is no FDA approved treatment for disease characterized by abnormal meibomian gland secretions. As such, there exists a need for an ocular therapeutic for normalizing meibomian gland secretions which is comfortable upon administration to the eye, eye lid, eye lashes and/or eye lid margin or a subject, and at a safe dose particularly suitable for long term use. The present invention meets this need and other needs.

### SUMMARY OF THE INVENTION

The invention features a method for the manufacture of a pharmaceutical composition for topical ophthalmic use by providing an amount of micronized minocycline and blending together the micronized minocycline with an amount of petrolatum to produce a composition having a final minocycline concentration of about 0.001% to 3.0%. Preferably, the final minocycline concentration is about 0.5% to 1.5%. Most preferably, the final minocycline concentration is about about 1%. The micronized minocyclne is jet-milled or cryo-milled. Preferably, the micronized minocycline has a diameter less than 20 μM. The petrolatum is at a concentration of about 50% to 100%. Optionally, the the micronized minocycline is sterilized prior to blending with the petrolatum. Suitable methods of sterilization include for example heat, moist heat, ethylene oxide (eto), or ionizing radiation. Ionizing radiation includes for example is gamma or e-beam.

Also include in the invention are pharmaceutical compositions produced by the method of manufacture according to the invention. In some embodiments the composition has less than about 1.0% epiminocycline after storage at room temperature for three months.

In other aspects, the invention provides a method of treating dry eye disease, comprising administering to a subject in need thereof the pharmaceutical composition produced according to the invention an amount effective to normalize meibomian gland secretions in the subject. The invention further includes methods of normalizing meibomian gland secretions, comprising administering to a subject in need thereof the pharmaceutical composition produced according to the invention in an amount effective to decrease the meibomian secretion viscosity, increase secretion transparency to a colorless state and decrease the time (refractory period) between gland secretions in the subject.

Other features and advantages of the invention will become apparent from the following detailed description and claims.

### BRIEF DESCRIPTION OF THE DRAWINGS

FIG. 1 is a table showing the stability of various ointment formulations with 3% minocycline.

FIG. 2 is a table showing the appearance of various formulations with 3% minocycline.

### DETAILED DESCRIPTION

For convenience, before further description of the present invention, certain terms employed in the specification, examples, and appended claims are collected here. These definitions should be read in light of the remainder of the disclosure and understood as by a person of skill in the art.

The term “abnormal meibomian gland secretion” refers to a meibomian gland secretion with increased viscosity, opacity, color and/or an increased time (refractory period) between gland secretions.

The term “aqueous” typically denotes an aqueous composition wherein the carrier is to an extent of >50%, more preferably >75% and in particular >90% by weight water.

The term “blepharitis” refers to a disorder comprising inflammation of the lid margin in which abnormal meibomian gland secretions plays a role and lid keratinization, lid margin rounding, obscuration of the grey line, increased lid margin transparency, and increased vascularity are observed. Although the terms meibomian gland dysfunction and meibomianitis are commonly referred to as blepharitis by most investigators, it is important to note that these are distinct diseases associated with abnormal meibomian gland secretions and that the terms are not interchangeable.

The term “comfortable” as used herein refers to a sensation of physical well being or relief, in contrast to the physical sensation of pain, burning, stinging, itching, irritation, or other symptoms associated with physical discomfort.

The term “comfortable ophthalmic formulation” as used herein refers to an ophthalmic formulation which provides physical relief from signs or symptoms associated with lid margin inflammation and/or ocular discomfort, and only causes an acceptable level of pain, burning, stinging, itching, irritation, or other symptoms associated with ocular discomfort, when instilled in the eye.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of lid margin irritation, or prevent or treat lid margin inflammation. The effective amount may vary depending on such factors as the disease or condition being treated, the particular composition being administered, or the severity of the disease or condition. One of skill in the art may empirically determine the effective amount of a particular agent without necessitating undue experimentation.

The phrase “pharmaceutically acceptable” is art-recognized and refers to compositions, polymers and other materials and/or salts thereof and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid (aqueous or non-aqueous) or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils such as castor oil, olive oil, peanut oil, macadamia nut oil, walnut oil, almond oil, pumpkinseed oil, cottonseed oil, sesame oil, corn oil, soybean oil, avocado oil, palm oil, coconut oil, sunflower oil, safflower oil, flaxseed oil, grapeseed oil, canola oil, low viscosity silicone oil, light mineral oil, or any combination thereof; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The term “preventing,” when used in relation to a condition, such as abnormal meibomian gland secretions, is art-recognized, and refers to administration of a composition which reduces the frequency of, or delays the onset of, signs and/or symptoms of a medical condition in a subject relative to a subject which does not receive the composition.

The term “treating” is an art-recognized term which refers to curing as well as ameliorating at least one symptom of any condition or disease.

1. Pharmaceutical Compositions and their Manufacture

The invention features topical ophthalmic pharmaceutical compositions comprising minocycline and a non-aqueous component (e.g., a pharmaceutically acceptable non-aqueous carrier). Surprisingly, it has been discovered viscosity of the non-aqueous carrier and particle size of the minocycline is important in the manufacture of the composition. In particular it has been demonstrated that the solubility, stability and potency of minocycline is superior in ointments as compared to other non-aqueous components such as oils. Additionally, micronization of the minocycline prior to formulation further improves the solubility, stability and potency. Accordingly, the invention provides a method for manufacture of pharmaceutical composition for topical ophthalmic containing micronized minocycline.

The viscosity of the composition and particle size of the minocycline is such as to optimize the bio-availability and to maintain the physical and chemical stability of the active ingredient. By optimize the bio-availability is meant that the viscosity of the composition is such that the composition remains at the site of application (e.g., lid margin) for a sufficient amount of time to allow the active ingredient to be absorbed. Physical stability is meant that the active ingredient remains uniformly suspended in the non-aqueous carrier. The composition is physically stable for at least 2, 4, 6 12, 18 or 24 months. Chemical stability is meant the rate in which the active ingredient degrades. Chemical stability is measured for example by the amount of impurities (e.g., epiminocycline) present in the composition over time. Preferably, the composition has less than about 2% epiminocycline, more preferably less than about 1% epiminocycline after storage at room temperature for three months.

Preferably, the non-aqueous component has viscosity of at least 5,000 cps, 7500 cps, 10,000 cps or greater. Optionally, other compounds may also be added to the formulations of the present invention to adjust (e.g., increase) the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Preferably, the non-aqueous component is an ointment. Preferred ointment base used to prepare the ophthalmic ointment of the present invention may be one that has been used in conventional ophthalmic ointments. In particular, the base may be liquid paraffin, white petrolatum, purified lanolin, gelation hydrocarbon, polyethylene glycol, hydrophilic ointment base, white ointment base, absorptive ointment base, Macrogol (Trade Name) ointment base, simple ointment base, and the like. Preferably, the non-aqueous component is an ointment having a petrolatum base. The ointment is at least 30%, petrolatum. Preferably, the ointment is 40%, 50% 60%, 70%, 75%, 80%, 85%, 90%, 95% or 100% petrolatum.

The optimal particle size of minocycline obtained by micronization. Minocycline is micronized by any suitable method known in the art. For example, all milling, grinding, micro-pulverization, controlled precipitation, jet-milling or cryo-milling. Preferably, the minocycline is jet-milled or cryo-milled. The particle size of the micronized minocycline is less that 20 μM, less than 15 μM, less than 10 μM, less than 5 μM.

Pharmaceutical ophthalmic formulations typically contain an effective amount, e.g., about 0.001% to about 10% wt/vol., preferably about 0.001% to about 5%, more preferably about 0.01% to about 3%, more preferably about 0.01% to about 1.5%, even more preferably about 0.001% to about 3%, even more preferably about 0.001% to about 1.5% of an active agent ingredient (e.g., minocycline) suitable for short or long term use for normalizing meibomian gland secretions. The amount of active ingredient will vary with the particular formulation and the disease state for which it is intended. In some embodiments the active agent is present in 0.05%, 0.06%, 0.07%, 0.08%, 0.09%, 0.1%, 0.2%, 0.3%. 0.4%, 0.5%, 0.6%, 0.7%. 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%. In some embodiments the active agent is present at about 1.0%

The active agents of the pharmaceutical compositions may be in the form of a pharmaceutically acceptable salt or free base.

Preferably, the effective amount of active agent present in the formulations should be sufficient to treat or prevent abnormal meibomian gland secretions. In certain embodiments, the active agent may treat or prevent abnormal meibomian gland secretions by normalizing (e.g., decreasing the meibomian secretion viscosity, increasing secretions transparency to a colorless state and decreasing the time (refractory period) between gland secretions) meibomian gland secretions.

An effective amount of the formulations of the invention may be used to normalize meibomian gland secretions, thereby treating diseases associated therewith (e.g., dry eye). Signs and symptoms of abnormal meibomian secretions include but are not limited to increased meibomian secretion viscosity, opacity, color, as well as an increase in the time (refractory period) between gland secretions. Signs and symptoms of diseases associated with abnormal meibomian gland secretions include but are not limited to dry eye, redness of the eyes, itching and/or irritation of the eyelid margins and edema, foreign body sensation, and matting of the lashes. Such formulations provide a comfortable ophthalmic formulation when instilled in the eye and have enhanced efficacy and duration of action over formulations of active agents (e.g., anti-infective agents) that are not combined with such non-aqueous components as described herein.

In certain embodiments, the compositions may treat or prevent abnormal meibomian gland secretions by normalizing meibomian gland function. (i.e., decreasing the meibomian secretion viscosity, increasing secretions transparency to a colorless state and decreasing the time (refractory period) between gland secretions).

The pharmaceutical compositions of the invention described above may additionally comprise other active ingredients, including, but not limited to, and vasoconstrictors, antiallergenic agents, anesthetics, analgesics, dry eye agents (e.g. secretagogues, mucomimetics, polymers, lipids, antioxidants), etc., or be administered in conjunction (simultaneously or sequentially) with pharmaceutical compositions comprising other active ingredients, including, but not limited to, and vasoconstrictors, antiallergenic agents, anesthetics, analgesics, dry eye agents (e.g. secretagogues, mucomimetics, polymers, lipids, antioxidants), etc.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N-(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenyhnercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

In another embodiment, the micronized minocycline and any other active ingredient is sterilized prior to formulation into an ointment. Sterilization is performed by dry heat, moist heat, eto, ionizing radiation (e.g., gamma or e-beam). After sterilization of the minocycline, the formulation is produced using aseptic processing.

Additional carriers may optionally be included in the formulations of the present invention. Examples of additional carriers include for example, water, mixtures of water and water-miscible solvents, such as C₁- to C₇-alkanols, vegetable oils, mineral oils or other oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, Nal, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Application may be performed with an applicator, such as the patient's finger, a Wek-Cel, Q-tip, or other device capable of delivering the formulation to the eye lid, eye lashes or eye lid margin in order to deliver the formulation to the meibomian gland orifice.

2. Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary. The formulations, if an ointment formulation, may be packaged as appropriate for an ointment, as is known to one of skill in the art.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and other publications by the Food and Drug Administration, and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

3. Methods of Use

The invention features methods of treating or preventing abnormal meibomian gland secretions in a subject comprising use of the novel formulations described above. For example, a method of treating or preventing abnormal meibomian gland secretions may comprise administering to the eye lid, eye lashes, or eye lid margin of a subject in need thereof a formulations according to the invention.

Such administration may reduce at least one sign of abnormal meibomian gland secretions in the subject and may operate by normalizing meibomian gland secretions in the subject.

The effective amount of active agent to include in a given formulation, and the efficacy of a formulation for normalizing meibomian gland secretions, may be assessed by one or more of the following: slit lamp evaluation, fluorescein staining, tear film breakup time, and evaluating meibomian gland secretions quality (by evaluating one or more of secretion viscosity, secretion color, gland alignment, vascularity pattern, vascularity redness, hyperkeratinization, posterior lid edge, lash, mucocutaneous junction, perigland redness, gland geometry and gland height).

The effective amount of active agent(s) in the formulation will depend on absorption, inactivation, and excretion rates of the drug as well as the delivery rate of the active agent(s) from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the composition and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

4. Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, ointments suspensions or formulations, tablets, or capsules of this invention. The kits can be designed to facilitate one or more aspects of shipping, use, and storage.

The kits may optionally include instructional materials containing directions (i.e., protocols) disclosing means of use of the formulations provided therein. The kits may also optionally include a topical applicator to facilitate administration of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

All publications and patents mentioned herein are hereby incorporated by reference in their entirety as if each individual publication or patent was specifically and individually indicated to be incorporated by reference. In case of conflict, the present application, including any definitions herein, will control.

### EXAMPLES

The invention now being generally described, it will be more readily understood by reference to the following examples which are included merely for purposes of illustration of certain aspects and embodiments of the present invention, and are not intended to limit the invention in any way.

### Example 1

### Minocycline Liquid Oil Formulations

Minocycline liquid oil formulations were produced as described in Table I. The stability of these formulations were assed at 6-months. Formulations were not stable at room temperature. 0.05% formulation decreased from 85.3% to 76.4% after 2 months storage at 25/60.

TABLE 1

| Formulation | Dose | Dosage | |
|---|---|---|---|
| Code | Strength | Form | Excipients |
| ACXMI-08-001 | 0.0% MI | Ophthalmic | 18.33% Olive Oil, NF; 73.33% |
| | | Solution | Castor Oil, USP; 8.33% |
| | | | Propylene Glycol, USP; |
| | | | 0.066% Magnesium Chloride, |
| | | | Hexahydrate, USP |
| ACXMI-08-002 | 0.03% MI | Ophthalmic | 18.33% Olive Oil, NF; 73.33% |
| | | Solution | Castor Oil, USP; 8.33% |
| | | | Propylene Glycol, USP; |
| | | | 0.066% Magnesium Chloride, |
| | | | Hexahydrate, USP |
| ACXMI-08-003 | 0.05% MI | Ophthalmic | 18.33% Olive Oil, NF; 73.33% |
| | | Solution | Castor Oil, USP; 8.33% |
| | | | Propylene Glycol, USP; |
| | | | 0.066% Magnesium Chloride, |
| | | | Hexahydrate, USP |

### Example 2

### Pre-formulation/Excipient Compatibility Studies

The objective of this study was to investigate excipient compatibility as part of pre-formulation development, with the goal of formulating Minocycline HCl in a liquid oil solution.

The solubility and stability of minocycline visually assessed in single-component phases. These phases single component phases include oils, surfactants and solvents. Oils included: mineral oil, olive oil and castor oil. Surfactants included: PEG 400 Monolaurate, Sorbitan Monolaurate, Tween 20, Tween 80, and Cremophor EL. Solvents included: PEG 200, and propylene glycol (with MgCl₂ stability additive) Solubility and stability was also evaluated in multi-component phases comprised of above excipients. Stability screen with Minocycline quantitated using HPLC analysis was performed with the most compatible excipient, mineral oil. The results of this study indicated that mineral Oil provided the greatest stability (least degree of epimerization) and was preferred relative to castor and olive oil due to minimal impurities being present in raw material. However, low solubility was achieved with mineral oil (NMT 0.02 mg/mL with preliminary assay method). It was concluded that formulation consisting of minocycline dissolved in an oil mixture was not feasible due to low solubility/stability.

### Example 3

### Feasibility of ‘Gelment’ (Low-Viscosity Ointment)

The purpose of this study was to investigate the feasibility of formulating Minocycline HCl in a ‘gelment’ (low-viscosity ointment).

Comfort was assessed for gelment prototypes ranging from 30% petrolatum/70% mineral oil concentration to 80% petrolatum/ 20% mineral oil. The results of this study indicated that Oil 30% petrolatum/70% mineral oil Mineral Oil was generally preferred from an overall comfort profile (resulting in least amount of blurriness).

Eye dropper bottles were not found to be a suitable container closure for the gelments selected. 0.05% Minocycline in up to 30% petrolatum was able to be dispensed from an eye drop bottle using an uncontrolled dropper tip. However, 0.1% Minocycline was difficult to dispense. Viscosity was found to increase with Minocycline concentration. Also, if samples were placed in colder room temperatures, the formulations became difficult to dispense.

### Example 4

### Feasibility of Medicated Swabs and Hydrophilic Excipient Bases

The purpose of this study was to investigate the feasibility of formulating Minocycline HCl in a ‘gelment’ with a medicated swab container closure. Specifically, formulation feasibility and stability with hydrophilic bases (PEG 400, propylene glycol, glycerol) investigated. Glycerin was unacceptable from a comfort perspective. PEG400 and propylene glycol did not provide sufficient stability, even with the addition of MgCl. (Following 3 weeks at 25/40, epi-minocycline was 20.7% for PEG 400 base and 2.9% for propylene glycol base.) Degradation was not detected following e-beam irradiation of Minocycline suspended in light mineral oil (single-point testing performed following irradiation—no stability evaluation).

### Example 5

### Feasibility of Mineral Oil Suspension Prototypical Formulation for POC

The purpose of this study was to investigate the feasibility of a mineral oil suspension for a prototypical formulation for evaluating in an initial clinical trial. Formulations were prepared as described in Table 2

TABLE 2

| Formulation Code | Dose Strength | Dosage Form | Excipients | Process Notes | Use |
|---|---|---|---|---|---|
| ACXMI-09-001 | 0.3% MI | Ophthalmic | 100% Penreco Light | Not homogenized | Stability/comfort |
| | | Suspension | Mineral Oil Drakeol 5 | | testing |
| ACXMI-09-002 | 0.0% MI | Ophthalmic | 100% Penreco Light | Not homogenized | Comfort testing |
| | | Solution | Mineral Oil Drakeol 5 | | |
| ACXMI-09-003 | 0.3% MI | Ophthalmic | 100% Penreco Light | Homogenized | Stability testing |
| | | Suspension | Mineral Oil Drakeol 5 | | |
| ACXMI-09-004 | 0.0% MI | Ophthalmic | 100% Methoxy | N/A | Stability/comfort |
| | | Solution | PEG350 NF | | testing |
| ACXMI-09-005 | 0.3% MI | Ophthalmic | 100% Methoxy | N/A | Stability/comfort |
| | | Solution | PEG350 NF | | testing |
| ACXMI-09-006 | 1% MI | Ophthalmic | 100% Penreco Light | Homogenized | Stability/comfort |
| | | Suspension | Mineral Oil Drakeol 5 | | testing |
| ACXMI-09-007 | 1% MI | Ophthalmic | 100% Penreco | Homogenized | Stability/comfort |
| | | Suspension | Ointment Base 4 | | testing |
| ACXMI-09-008 | 0.1% MI | Ophthalmic | 100% Penreco Light | Homogenized | Stability testing |
| | | Suspension | Mineral Oil Drakeol 5 | | |
| ACXMI-09-009 | 0% MI | Oil | 100% Penreco Light | N/A | Zone of inhibition |
| | | | Mineral Oil Drakeol 5 | | testing |
| ACXMI-09-010 | 0.1% MI | Ophthalmic | 100% Penreco Light | Homogenized | Zone of inhibition |
| | | Suspension | Mineral Oil Drakeol 5 | | testing |
| | | | (repeat of ACXMI-09-008) | | |
| ACXMI-09-011 | 0% MI | Ophthalmic | 100% Penreco | N/A | Zone of inhibition |
| | | Ointment | Ointment Base 4 | | testing |
| ACXMI-09-012 | 0.1% MI | Ophthalmic | 100% Penreco | Homogenized | N/A - prepared |
| | | Ointment | Ointment Base 4 | | for potential |
| | | | | | comfort/ZOI use |
| ACXMI-09-013 | 3% MI | Ophthalmic | 100% Penreco | Homogenized | N/A - prepared |
| | | Ointment | Ointment Base 4 | | for potential |
| | | | | | comfort/ZOI use |
| ACXMI-09-014 | 1% MI | Ophthalmic | 100% Penreco | Homogenized | Zone of inhibition |
| | | Ointment | Ointment Base 4 | | and comfort testing |
| ACXMI-09-015 | 0.25% MI | Ophthalmic | 100% Penreco | Homogenized | N/A - prepared |
| | | Ointment | Ointment Base 4 | | for potential |
| | | | | | comfort/ZOI use |
| ACXMI-09-016 | 0.5% MI | Ophthalmic | 100% Penreco | Homogenized | N/A - prepared |
| | | Ointment | Ointment Base 4 | | for potential |
| | | | | | comfort/ZOI use |

Physical stability was not achieved with 100% mineral oil suspensions (sample analysis yielded highly variable minocycline recoveries at T0, even with homogenization) Chemical stability not achieved when surfactants were added to aid with settling and re-dispersion. MPEG350 did not provide superior stability over PEG 400. Ointment formulation provided on-target assay recoveries at T0 but Minocycline HCl decreased over time (67.3-85.1% LC at 4 weeks; 33.0-37.3% LC at 2 months). Significant epimerization was not observed, however, suggesting the formulation was chemically stable. 1% Minocycline HCl (unmicronized) ointment was determined to be comfortable (comfort scores<3) over a 3-day QD dosing period (n-6). Mineral oil and ointment formulations had comparable or higher zones of inhibition relative to Vancomycin.

### Example 6

### Feasibility of Micronized Minocycline HCl Ointment and Minocycline Free Base in Oil Formulations

The purpose of this study was to investigate the feasibility of a hydrocarbon-based (mineral oil/petrolatum) ointment formulation with micronized Minocycline HCl or minocycline free base in an oil solution formulation. Formulations were prepared as described in Table 3.

TABLE 3

| Formulation Code | Dose Strength | Dosage Form | Excipients | Process Notes | Use |
|---|---|---|---|---|---|
| ACXMI-09-017 | 3.0% | Opthalmic | 3% Cryomilled Minocycline | Cryomilled | Stability testing |
| | | Suspension | 97% Light Mineral Oil, NF | Minocycline, | |
| | | | Penrco Drakeol 5 Lt | homogenized | |
| ACXMI-09-018 | 3.0% | Opthalmic | 3% Jetmilled Minocycline | Jetmilled | Stability testing |
| | | Suspension | 97% Light Mineral Oil, NF | Minocycline, | |
| | | | Penrco Drakeol 5 Lt | homogenized | |
| ACXMI-09-019 | 3.0% | Opthalmic | 3% Unmicronized Minocycline | Homogenized | Stability testing |
| | | Suspension | 97% Light Mineral Oil, NF | | |
| | | | Penrco Drakeol 5 Lt | | |
| ACXMI-09-020 | 3.0% | Ophthalmic | 100% Sonneborn | Cryomilled | Stability testing |
| | | Ointment | White Protopet 1S, USP | Minocycline. | |
| | | | | Prepared by DPT. | |
| ACXMI-09-021 | 3.0% | Ophthalmic | 100% Sonneborn | Jetmilled | Stability and |
| | | Ointment | White Protopet 1S, USP | Minocycline. | comfort testing |
| | | | | Prepared by DPT. | |
| ACXMI-09-022 | 3.0% | Ophthalmic | 100% Sonneborn | Unmicronized | Stability and |
| | | Ointment | White Protopet 1S, USP | Minocycline. | comfort testing |
| | | | | Prepared by DPT. | |
| ACXMI-09-023 | 3.0% | Ophthalmic | 85% Sonneborn | Jetmilled | Stability testing |
| | | Ointment | White Protopet 1S, USP; | Minocycline. | |
| | | | 15% Mineral Oil, USP | Prepared by DPT | |
| ACXMI-09-024 | 3.0% | Ophthalmic | 75% Sonneborn | Jetmilled | Stability testing |
| | | Ointment | White Protopet 1S, USP; | Minocycline. | |
| | | | 25% Mineral Oil, USP | Prepared by DPT | |

Two Minocycline HCl drug substance batches were micronized (by jetmilling and cryomilling) and evaluated for chemical properties post-micronization. Jetmilled, cryomilled, and unmicronized oil and ointment formulations were evaluated for batch uniformity and stability.

Oil formulations were 100% oil and ointments were 100% petrolatum. Additional jetmilled Minocycline HCl ointment formulations with lower viscosity (85/15 and 75/25) were prepared evaluated for batch uniformity and stability. Minocycline free base evaluated with respect to solubility in light mineral oil.

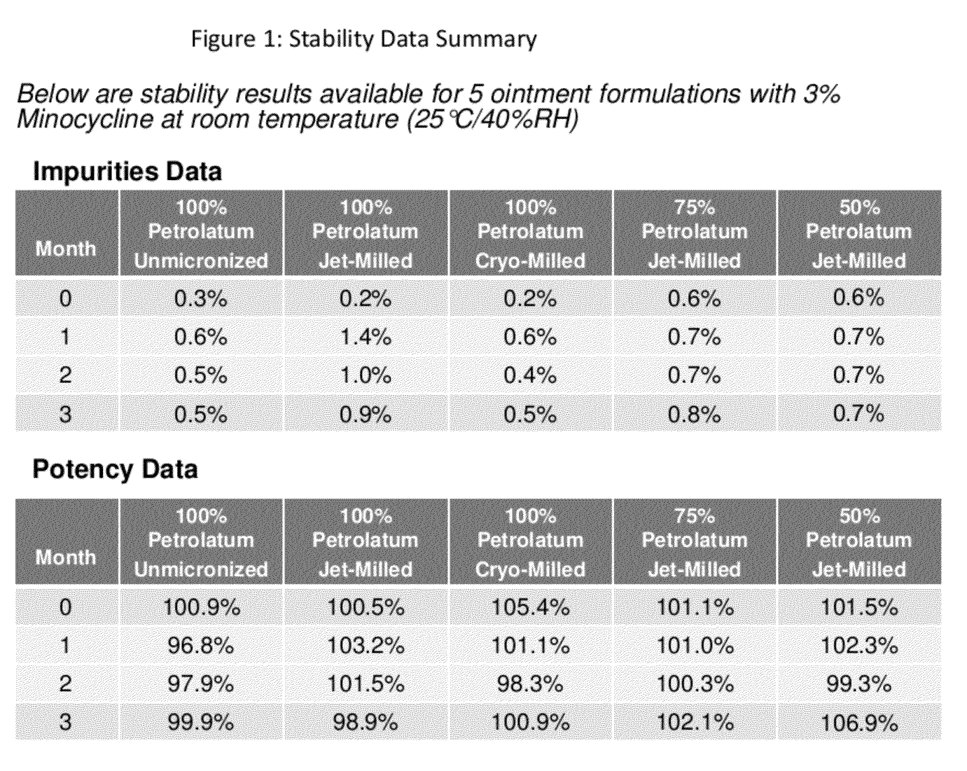

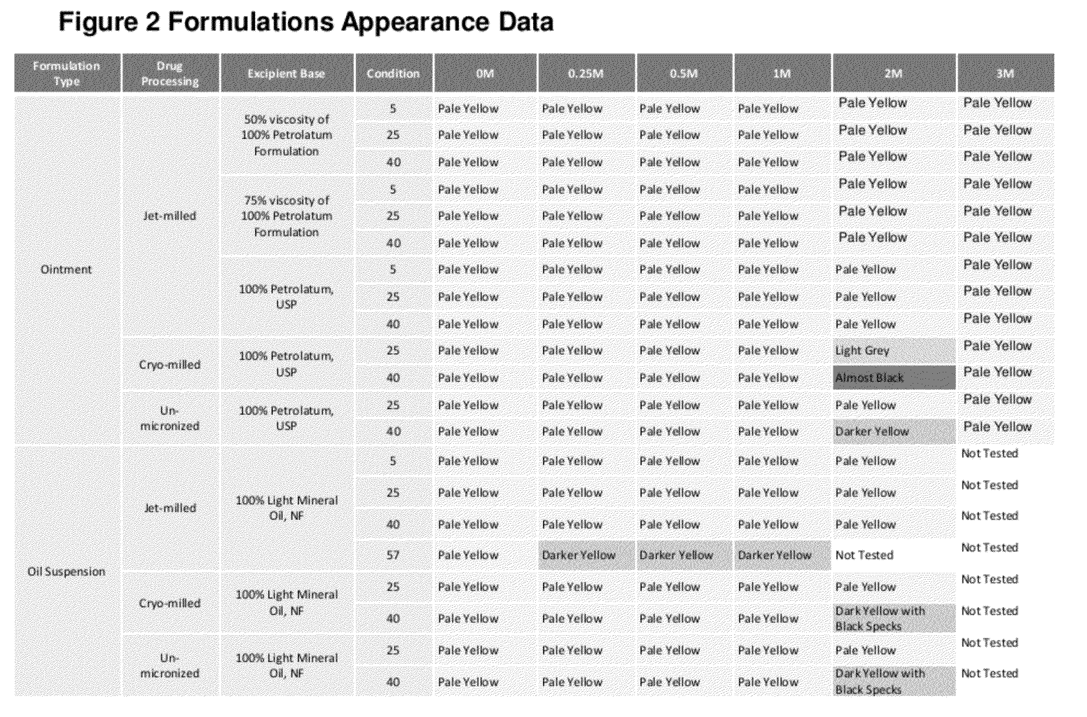

Stability data is shown in FIG. 1. Formulations appearance data is shown in FIG. 2.

No change in assay was found following micronization (jetmilling or cryomilling) for 2 batches of Minocycline HCl drug substance. The oil suspensions in this study provided consistent T0 results, unlike the earlier oil formulations with unmicronized Minocycline HCl. The stability data did not indicate settling for either the jetmilled, cryomilled, or unmicronized oil suspensions, however evaluation was discontinued following the 2M time-point due to appearance changes. Ointment stability data through 3M suggested that the formulations would be stable for 24M at least refrigerated conditions, if not room temperature.

The stability data suggested that jet-milling resulted in higher epiminocycline levels relative to cryomilling and no micronization.

The Minocycline free base was not highly soluble in oil.

It was concluded that ointment formulation could be further developed and should have at least a 24M shelf-life at refrigerated conditions (if not room temperature).

### Equivalents

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

### REFERENCES

All publications and patents mentioned herein are hereby incorporated by reference in their entireties as if each individual publication or patent was specifically and individually indicated to be incorporated by reference. In case of conflict, the present application, including any definitions herein, will control.

Sullivan, D. A. et al. (2000) Invest. Ophthalmol. Vis. Sci. 41(12):3732-3742.

Mathers, W. D. Meibomian Gland Disease. In: Pflugelder, S. et al, editors. Dry Eye and Ocular Surface Disorders. Marcel Dekker, Inc. New York.

Bron, A. J., et al. (1991) Eye 5:395-411.

Cassin, et al. Dictionary of Eye Terminology. 4^{th} Ed. Gainesville, Fla. Triad Communications, Inc. (2001).

What is claimed is:

1. A method for the manufacture of a pharmaceutical composition for topical ophthalmic use comprising:
a) providing an amount of micronized minocycline;
b) blending together the minocycline of step (a) with an amount of petrolatum to produce a composition having a final minocycline concentration of about 0.001% to 3.0%.

2. The method of claim 1, wherein the minocyclne is jet-milled or cryo-milled.

3. The method of claim 1, wherein the petrolatum is at a concentration of about 50% to 100%.

4. The method of claim 1, wherein the final minocycline concentration is about 0.5% to 1.5%.

5. The method of claim 1, wherein the final minocyline concentration is about 1%.

6. The method of claim 1, wherein the micronized minocycline is sterilized prior to step (b).

7. The method of claim 6, wherein the micronized minocycline is sterilized by heat, moist heat, ethylene oxide (eto), or ionizing radiation.

8. The method of claim 7, wherein the ionizing radiation is gamma or e-beam.

9. The method of claim 1, wherein the micronized minocycline has a diameter less than 20 μM.

10. The pharmaceutical composition produced by the method of claim 1.

11. The composition of claim 10, wherein said composition has less than about 1.0% epiminocycline after storage at room temperature for three months.

12. A method of treating dry eye disease, comprising administering to a subject in need thereof the pharmaceutical composition of claim 10 in an amount effective to normalize meibomian gland secretions in the subject.

13. A method of normalizing meibomian gland secretions, comprising administering to a subject in need thereof the pharmaceutical composition of claim 10 in an amount effective to decrease the meibomian secretion viscosity, increase secretion transparency to a colorless state and decrease the time (refractory period) between gland secretions in the subject.
